(19) United States
(12) Patent Application Publication
(10) Pub. No.: US 2013/0251690 A1
(43) Pub. Date: Sep. 26, 2013

(54) STEM CELL DIFFERENTIATION USING KERATIN BIOMATERIALS

(71) Applicant: Wake Forest University Health Sciences, Winston-Salem, NC (US)

(72) Inventors: Mark E. Van Dyke, Winston-Salem, NC (US); Thaleia Teli, Athens (GR)

(73) Assignee: Wake Forest University Health Sciences, Winston-Salem, NC (US)

(21) Appl. No.: 13/895,599

(22) Filed: May 16, 2013

Related U.S. Application Data
(63) Continuation of application No. PCT/US2011/061194, filed on Nov. 17, 2011.
(60) Provisional application No. 61/414,751, filed on Nov. 17, 2010.

(51) Int. Cl. C12N 5/071

(52) U.S. Cl. CPC C12N 5/0654
USPC 424/93.7; 435/377; 435/366

(57) ABSTRACT

Provided are methods and keratin compositions useful for modulation of cell differentiation, both in vitro and in vivo, for example, in cell therapy applications.

### RELATED APPLICATIONS

This application claims the benefit of U.S. Provisional Patent Application Ser. No. 61/414,751, filed Nov. 17, 2010, the disclosure of which is incorporated by reference herein in its entirety.

### FIELD OF THE INVENTION

The present invention is generally related to the use of keratin-based biomaterials for modulation of cell differentiation, both in vivo and in vitro.

### BACKGROUND OF THE INVENTION

Regenerative medicine is a promising approach for the repair of tissue deficiencies caused by trauma, degenerative diseases, and tumors. For patients with bone or cartilage injury or disease, such as fractures, osteoporosis, arthritis, or cancer, the combination of autologous, multipotent mesenchymal stem cells and a biomaterial carrier has been proposed as a treatment. In the adult and elderly patient population, however, significant changes in the multipotency of mesenchymal stem cells with age have brought the clinical utility of this treatment paradigm into question.

One mechanism which may contribute to this change in multipotency involves the change in expression of peroxisome proliferator-activated receptor gamma 2 (PPARγ2 or PPARγ). PPARγ2 has been shown to activate adipogenic and suppress osteogenic differentiation pathways in aged mice, which limits the potential effectiveness of regenerative treatments that use endogenous and exogenous mesenchymal stem cells for bone or cartilage repair.

Alternative regenerative medicine strategies useful for bone or cartilage repair are therefore needed, particularly when using cells that may have a reduced capacity to differentiate into these tissues.

### SUMMARY OF THE INVENTION

Provided are methods of treating a bone injury or disease in a subject in need thereof (e.g. a human subject), including administering in combination to the subject in a treatment effective amount: i) a composition comprising a keratin; and ii) a composition comprising stem cells or progenitor cells.

In some embodiments, the keratin is selected from the group consisting of: keratose, kerateine, KAP, and combinations thereof. In some embodiments, the keratin is selected from the group consisting of: acidic keratose, acidic kerateine, KAP, and combinations thereof. In some embodiments, the keratin is selected from the group consisting of: alpha/KAP keratose, gamma keratose, alpha/KAP kerateine, gamma kerateine, and combinations thereof.

In some embodiments, the cells include menenshymal stem cells (e.g., harvested from bone marrow or adipose tissue) or adipose derived stem cells.

In some embodiments, the cells are harvested from an adult or elderly donor. In some embodiments, the cells are autologous or allogeneic. In some embodiments, the donor is a human donor who is at least 40, 45, 50, 55, 60, 65, 70 or 75 years old at the time of harvest. In some embodiments, the donor is a human donor who is at least 60, 65, 70, 75 or 80 years old at the time of harvest.

Further provided are methods of differentiating stem cells into osteogenic cells, including: providing stem cells; providing a keratin in contact (e.g., fluid contact) with the stem cells; and providing conditions conducive to differentiation of the stem cells into osteogenic cells, to thereby differentiate the stem cells into osteogenic cells.

In some embodiments, the keratin is selected from the group consisting of: keratose, kerateine, KAP, and combinations thereof. In some embodiments, the keratin is selected from the group consisting of: acidic keratose, acidic kerateine, KAP, and combinations thereof. In some embodiments, the keratin is selected from the group consisting of: alpha/KAP keratose, gamma keratose, alpha/KAP kerateine, gamma kerateine, and combinations thereof.

In some embodiments, the cells include menenshymal stem cells (e.g., harvested from bone marrow or adipose tissue) or adipose derived stem cells.

In some embodiments, the cells are harvested from an adult or elderly donor. In some embodiments, the cells are autologous or allogeneic. In some embodiments, the donor is a human donor who is at least 40, 45, 50, 55, 60, 65, 70 or 75 years old at the time of harvest. In some embodiments, the donor is a human donor who is at least 60, 65, 70, 75 or 80 years old at the time of harvest.

Still further provided are methods of differentiating stem cells into chondrogenic cells, including: providing stem cells; providing a keratin in contact (e.g., fluid contact) with the stem cells; and providing conditions conducive to differentiation of the stem cells into chondrogenic cells, to thereby differentiate the stem cells into osteogenic cells.

In some embodiments, the keratin is selected from the group consisting of: keratose, kerateine, KAP, and combinations thereof. In some embodiments, the keratin is selected from the group consisting of: acidic keratose, acidic kerateine, KAP, and combinations thereof. In some embodiments, the keratin is selected from the group consisting of: alpha/KAP keratose, gamma keratose, alpha/KAP kerateine, gamma kerateine, and combinations thereof.

In some embodiments, the cells include menenshymal stem cells (e.g., harvested from bone marrow or adipose tissue) or adipose derived stem cells.

In some embodiments, the cells are harvested from an adult or elderly donor. In some embodiments, the cells are autologous or allogeneic. In some embodiments, the donor is a human donor who is at least 40, 45, 50, 55, 60, 65, 70 or 75 years old at the time of harvest. In some embodiments, the donor is a human donor who is at least 60, 65, 70, 75 or 80 years old at the time of harvest.

Also provided is the use of a keratin as provided herein for treating a bone or cartilage injury or disease, and the use of a keratin as provided herein for the preparation of a medicament for treating the same.

### BRIEF DESCRIPTION OF THE DRAWINGS

FIG. 1. Surface marker characterization human adipose-derived stem cells (ADSC). Characterization of ADSC. Flow cytometry histograms of ADSC at passage 2 show the expression (shaded) of the selected surface marker.

FIG. 2. Gene expression in differentiated ADSC. These large fold-changes in osteogenic genes are indicative of improved differentiation of ADSC when treated with keratin.

FIG. 3. Gene expression of PPARγ in ADSC treated with keratin. All keratin fractions elicit a down-regulation of PPARγ expression, particularly. This keratin-induced PPARγ down-regulation at early time points promotes the ADSC towards osteogenic fate; it is sustained throught and is particularly profound at later time points, suggesting terminal osteogenic differentiation.

FIG. 4. Alkaline Phosphatase activity on Day 14.

FIG. 5. Alizarin red staining for calcium deposition. All fractions of keratin induce more calcified matrix deposition than the positive control, dexamethisone (shown as DEX 10-7).

FIG. 6. Western blot analysis of media. These data confirm the production of osteocalcin protein and validate the gene expression analysis. Panel A shows the antibody staining of separated proteins and panel B is quantitative data of relative levels of protein content in samples treated with gamma-keratose.

FIG. 7. Bone marrow derived MSC differentiation on keratin and control substrates.

FIG. 8. Adipose derived MSC differentiation on keratin and control substrates.

### DETAILED DESCRIPTION OF THE PREFERRED EMBODIMENTS

Described herein are keratin compositions useful in modulating cell differentiation, e.g., the differentiation of stem or progenitor cells. According to some embodiments, the keratin compositions provided herein cause cells (e.g., stem or progenitor cells) to express genes indicative of a bone cell phenotype, secrete a mineralized matrix, and/or suppress the expression of the gene for the transcription factor peroxisome proliferator-activated receptor gamma (PPARγ), a regulator of adipogenesis. The ability of keratins to regulate the fate of stem cells such as mesenchymal stem cells (MSC) and the ability to enhance osteogenic or chondrogenic differentiation is useful to improve the capability of these cells to serve as a readily available source of stem cell-derived osteoblasts or chondrocytes, respectively, for research or therapy, particularly those cells harvested from adult or elderly donors (e.g., autologous stem cells used in an adult or elderly patient).

The disclosures of all cited United States Patent references are hereby incorporated by reference to the extent they are consistent with the disclosure herein. As used herein in the description and the appended claims, the singular forms “a,” “an” and “the” are intended to include the plural forms as well, unless the context clearly indicates otherwise. Furthermore, the terms “about” and “approximately” as used herein when referring to a measurable value such as an amount of a compound, dose, time, temperature, and the like, is meant to encompass variations of 20%, 10%, 5%, 1%, 0.5%, or even 0.1% of the specified amount. Also, as used herein, “and/or” refers to and encompasses any and all possible combinations of one or more of the associated listed items, as well as the lack of combinations when interpreted in the alternative (“or”).

Keratins are a family of proteins found in the hair, skin, and other tissues of vertebrates. Hair is a unique source of human keratins because it is one of the few human tissues that is readily available and inexpensive. Although other sources of keratins are acceptable feedstocks for the present invention, (e.g. wool, fur, horns, hooves, beaks, feathers, scales, and the like), human hair is preferred for use with human subjects because of its biocompatibility. The human hair can be end-cut, as one would typically find in a barber shop or salon.

“Keratin derivative” as used herein refers to any keratin fraction, derivative, subfamily, etc., or mixtures thereof, alone or in combination with other keratin derivatives or other ingredients, including, but not limited to, alpha keratose, gamma keratose, alpha kerateine, gamma kerateine, keratin-associated proteins (KAP), and combinations thereof, including the acidic and basic constituents thereof unless specified otherwise, along with variations thereof that will be apparent to persons skilled in the art in view of the present disclosure.

### I. Cells

“Cell” or “cells” are preferably mammalian cells (including mouse, rat, dog, cat, monkey and human cells), and in some embodiments human cells are preferred. “Isolated” as used herein signifies that the cells are placed into conditions other than their natural environment. Tissue or cells are “harvested” when initially isolated from a donor, e.g., a primary explant. “Cell culture” is the growth or proliferation of cells in vitro. Cells include, but are not limited to, stem and progenitor cells (whether embryonic, fetal, or adult), germ cells, somatic cells, cells strains or cell lines, etc., without limitation (See, e.g., U.S. Pat. No. 6,808,704 to Lanza et al.; U.S. Pat. No. 6,132,463 to Lee et al.; and U.S. Patent Application Publication No. 2005/0124003 to Atala et al.). The cell donor may be of any age, including newborn, neonate, infant, child, adolescent, adult, and elderly.

When a culture system includes factors that induce differentiation in cells, especially differentiation into bone or chondrocyte cells, in some embodiments a higher yield of the terminal cell type is achieved with the use of keratin biomaterials as described herein (e.g., by 10, 20, 30, 40, 50, 60, 70, 80, 90 or 100% or more). Differentiation of cells into osteoblasts or chondrocytes, for example, may be performed as known in the art, e.g., using media additives such as dexamethisone, beta-glycerol phosphate, vitamin C, and/or growth factors such as bone morphogenetic proteins, etc. (see, e.g., Pittenger MF et al. Multilineage potential of adult human mesenchymal stem cells. Science 1999; 284(5411):143-7).

Cells may be identified and/or categorized by methods known in the art, e.g., based upon properties that distinguish one cell type from another, e.g., density, size, shape, unique markers, unique metabolic pathways, nutritional requirements, protein expression, protein excretion, etc. Unique markers may be selected with fluorescent activated cell sorting (FACS), immunomagnetic bead sorting, magnetic activated cell sorting (MACS), panning, etc. Unique metabolic pathways and nutritional requirements may be assessed by varying the makeup and/or quantity of nutritional ingredients of the medium on which cells are grown, particularly in a serum-free environment. Protein expression and/or excretion may be detected with various assays, e.g., ELISA.

“Mesenchymal stem cells” or “MSC” as used herein refers to cells that are characterized by their ability to differentiate into bone, cartilage, or fat (i.e., adipose) cells. In some embodiments, MSC are plastic-adherent when maintained in standard culture conditions. In some embodiments, MSC express markers CD105, CD73 and CD90. In some embodiments, MSC lack expression of markers CD45, CD34, CD14 or CD11b, CD79a or CD19, and HLA-DR. See, e.g., Dominici et al., “Minimal criteria for defining multipotent mesenchymal stromal cells. The International Society for Cellular Therapy position statement,” Cytotherapy (2006): 8(4): 315-317. Mesenchymal stem cells are normally found in, and may be isolated from, e.g., adipose tissue or bone marrow, or from other tissues such as peripheral blood, blood vessel, skeletal muscle, skin, teeth, and gut.

“Bone marrow-derived stem cells” or “BMSC” are MSC harvested from bone marrow tissue. In some embodiments, BMSC include adherent marrow stromal cells.

“Adipose-derived stem cells” or “ADSC” are MSC harvested from adipose tissue. In some embodiments, ADSC express CD13, CD44, CD73, CD105, CD106, and/or vimentin. In some embodiments, ADSC are negative for the hematopoietic markers CD14 and CD45. ADSC may be collected from, e.g., adipose depots such as the abdominal superficial, the thigh, the abdominal deep, the arm, the thigh and/or the trochanteric depot.

“Bone cells” are those cells normally found in bone, and include osteoblasts, osteoclasts, osteocytes, and any combination thereof. Bone cells cultured using the processes described herein are useful for, among other things, implantation into a subject to treat bone fractures or defects, and/or promote bone healing, resulting from, e.g., injury or disease (e.g., osteoporosis, osteogenesis imperfecta, Paget's disease of bone, cancer, etc.).

Bone injury or disease include those associated with tissue damage (e.g., following surgery), and an imbalance in the regulation of bone formation and resorption, particularly those diseases characterized by a net bone loss (bone resorption exceeds bone formation), and include, but are not limited to, ostopenia, osteoporosis and osteolysis, including, but not limited to, primary osteoporosis, endocrine osteoporosis (including, but not limited to, hyperthyroidism, hyperparathyroidism, Cushing's syndrome, and acromegaly, hereditary and congenital forms of osteoporosis (including, but not limited to, osteogenesis imperfecta, homocystinuria, Menkes' syndrome, Riley-Day syndrome)), osteoporosis due to immobilization of extremities, glucocorticoid-induced osteoporosis and post-menopausal osteoporosis, (juvenile or familial) Paget's disease, osteomyelitis (i.e., an infectious lesion in bone leading to bone loss), osteopenia following surgery, osteopenia induced by steroid administration, osteopenia associated with disorders of the small and large intestine, and osteopenia associated with chronic hepatic and renal diseases, osteonecrosis (i.e., bone cell death), including, but not limited to, osteonecrosis associated with traumatic injury, osteonecrosis associated with Gaucher's disease, osteonecrosis associated with sickle cell anemia, osteonecrosis associated with systemic lupus erythematosus, osteonecrosis associated with rheumatoid arthritis, osteonecrosis associated with periodontal disease, osteonecrosis associated with osteolytic metastasis, and osteonecrosis associated with other condition, bone loss associated with arthritic disorders such as psoriatic arthritis, rheumatoid arthritis, loss of cartilage and joint erosion associated with arthritis, including inflammatory arthritis (e.g., rheumatoid arthritis), collagen-induced arthritis, periprosthetic osteolysis, etc., bone loss associated with aromatase inhibitor therapy, bone loss associated with androgen deprivation therapy, bone loss associated with bone metastasis, bone loss associated with diseases having immune system involvement, such as adult and childhood leukemias, cancer metastasis, autoimmunity, and various viral infections.

“Cartilage cells” include those cells normally found in cartilage, which cells include chondrocytes. Chondrocytes produce and maintain the extracellular matrix of cartilage, by, e.g., producing collagen and proteoglycans. Cartilage is a highly specialized connective tissue found throughout the body, and its primary function is to provide structural support for surrounding tissues (e.g., in the ear and nose) or to cushion (e.g., in the trachea and articular joints). Types of cartilage include hyaline cartilage (articular joints, nose, trachea, intervertebral disks (NP), vertebral end plates), elastic cartilage (tendon insertion site, ligament insertion site, meniscus, intervertebral disks (AP)), costochondral cartilage (rib, growth plate), and fibrocartilage (ear).

The loss of cartilage in a subject can be problematic, as it has a very limited repair capacity. Cartilage cells cultured using the processes described herein are useful for, among other things, implantation into a subject to treat cartilage injury or disease, or for cosmetic surgery applications.

“Adipose cells” include those cells normally found in fat tissue (e.g., white fat tissue or brown fat tissue), which cells include adipocytes and preadipocytes.

“Peroxisome proliferator-activated receptor gamma” or “PPARγ” is considered a regulator of adipogenesis. High PPARγ expression favors differentiation of MSC to adipocytes; conversely, PPARγ insufficiency favors differentiation of MSC to osteoblasts or chondrocytes. See Akune et al., J Clin Invest 2004; 113(6):846-55.

It has been shown that PPARγ expression naturally increases in MSC harvested from older donors (see Moerman et al., Aging Cell 2004; 3(6):379-89), thereby biasing these cells to form adipocytes rather than osteoblasts or chondrocytes, even when well-established differentiation protocols are used. Therefore, down-regulation of the expression of PPARγ may enhance the differentiation of stem and progenitor cells toward an osteogenic or a chondrogenic fate. Keratin biomaterials according to some embodiments can achieve this, and therefore can serve as adjuvants in the process of stem and progenitor cell differentiation, creating a more efficient production of the desired cell phenotype, in vitro and/or in vivo.

In some embodiments, osteocalcin expression upon differentiation shows a fold-change of >1 as compared to an undifferentiated control, and preferably >2 or >5, In some embodiments, PPARγ expression is reduced to <1, <½, or < 1/20, as compared to an undifferentiated control.

Subjects are generally human subjects and include, but are not limited to, “patients.” The subjects may be male or female and may be of any race or ethnicity, including, but not limited to, Caucasian, African-American, African, Asian, Hispanic, Indian, etc. The subjects may be of any age, including newborn, neonate, infant, child, adolescent, adult, and elderly.

Subjects also include animal subjects, particularly mammalian subjects such as canines, felines, bovines, caprines, equines, ovines, porcines, rodents (e.g., rats and mice), lagomorphs, non-human primates, etc., for, e.g., veterinary medicine, laboratory research and/or pharmaceutical drug development purposes.

“Treat” refers to any type of treatment that imparts a benefit to a patient, e.g., a patient having defect in a bone or cartilage tissue, which may be due to injury, disease, etc. Treating includes actions taken and actions refrained from being taken for the purpose of improving the condition of the patient (e.g., the relief of one or more symptoms), delay in the onset or progression of the injury or disease, etc.

Treatment for a bone injury such as a fracture or nonunion may be performed by bone grafting, and may or may not include bone cells in the graft. Autologous bone cells may be obtained, for example, from the patient's bone tissue (e.g., from the pelvis, the iliac crest, the chin, the fibula, the ribs, the mandible, the skull, etc.). Allogeneic bone may also be used (e.g., from a cadaveric donor). In some embodiments, the bone cells or tissue may be intermixed with the keratin-containing compositions taught herein.

Assessment of bone fracture or nonunion may be conducted as known in the art, e.g., x-ray. Assessment of bone density may be conducted as known in the art, e.g., dual energy x-ray absorptiometry, computer-assisted tomography and transmission ultrasound, etc. See U.S. Patent Application Publication No. 2010/0113932. In some embodiments, and as desired, treatment may be ongoing until bone density or union is demonstrated, e.g., by x-ray or CT data, mineralization as defined by a >50, 60, 70, 80 or 90% normal mineral density, limb stability and load bearing as demonstrated by patient exam, etc.

With respect to the subject to be treated, cells may be syngeneic (i.e., genetically identical or closely related, so as to minimize tissue transplant rejection), allogeneic (i.e., from a non-genetically identical member of the same species) or xenogeneic (i.e., from a member of a different species) with respect to a subject. Syngeneic cells include those that are autogeneic (i.e., from the patient to be treated) and isogeneic (i.e., a genetically identical but different subject, e.g., from an identical twin). Cells may be obtained from a donor (either living or cadaveric) or derived from an established cell strain or cell line. For example, cells may be harvested from a donor (e.g., a potential recipient of a bioscaffold graft) using standard biopsy techniques known in the art.

In some embodiments, cells are harvested from a donor who is an adult at the time of harvest. As used herein, an “adult” means the donor is physically mature. An adult human, for example, according to some embodiments is at least 25, 30, 35, 40, 45, 50, or 55 years of age. The adult human donor according to some embodiments may also be older or elderly, for example, at least 60, 65, 70, 75, 80, 85 or 90 years of age at the time of harvest.

According to some embodiments, the keratin compositions described herein may be used as additives to cell culture media. The compositions may also be used in coatings, gels, three-dimensional scaffolds, microcarriers, etc. See U.S. Patent Application publication no. 2010/0197021 to Van Dyke, which is incorporated by reference herein. In some embodiments, the keratin is provided in a concentration of from 0.0001, 0.005, or 0.01 to 0.05, 0.1, or 0.5 mg/mL (e.g., as an additive in the culture media). In some embodiments, the keratin is provided in a concentration of from 0.01 to 0.03 mg/mL

In some embodiments, cells are provided in or further include a liquid carrier. The liquid carrier can be in the form of a suspension, solution, or any other suitable form, and may or may not include a keratin derivative as described herein. Examples of suitable liquid carriers include, but are not limited to, water, aqueous solutions (e.g., phosphate buffer solution, citrate buffer solution, etc.), liquid media (e.g., modified Eagle's medium (“MEM”), Hanks' Balanced Salts, etc.), gels (e.g., hydrogels), and so forth, and in some embodiments may include additional ingredients, as desired.

The keratin compositions or substrates in some embodiments may be used to deliver cells in cell therapy applications. The keratin may also be provided as an implant, providing a matrix for endogenous or exogenous cells to infiltrate, differentiate and form tissue. Implants include keratins in the form of gels, coatings, fibers, scaffolds, etc., such as a nonwoven mesh, sponge, or hydrogel. Without wishing to be bound by theory, once stem or progenitor cells infiltrate the implant and come in contact with the keratin, gene expression changes are thought to occur due at least in part to the presence of the keratin, which promotes differentiation to form tissues of interest. In some embodiments, stem or progenitor cells may be mixed with or seeded onto the keratin (with or without other differentiation factors) and placed into a tissue defect.

Proteins (such as growth factors) or other additives (such as differentiation factors, antibiotics, anti-inflammatories, and modulators of the immune response) may also be added to the cell and/or keratin preparations at any time. Also, various treatments may be applied to enhance adherence of cells to a substrate and/or to each other. Appropriate treatments are described, for example, in U.S. Pat. No. 5,613,982. For example, collagen, elastin, fibronectin, laminin, or proteoglycans may be included. As used herein, “growth factors” include molecules that promote the regeneration, growth and survival of cells or tissue. Growth factors may be those naturally found in keratin extracts, or may be in the form of an additive, such as bone-derived growth factors, nerve growth factor (NGF), aFGF, bFGF, PDGF, TGFβ, VEGF, GDF-5/6/7, bone morphogenetic protein (e.g., BMP-1/2/3/4/5/6/7/8a/8b/10/13/12/14/15), IGF-1, etc.

### II. Preparation of Keratin Solutions and Substrates

As noted above, one source of keratins is human hair, which may be end-cut as one would typically find in a barbershop or salon, or purchased through commercial sources. It can be cleaned by washing in a warm water solution of mild detergent and freed of surface oils by washing with an organic solvent such as ethanol, ether, or acetone. A preferred solvent is ethanol. Soluble keratins can be extracted from human hair fibers by oxidation or reduction using methods known in the art (see, for example, Rouse J G, Van Dyke M E. A review of keratin-based biomaterials for biomedical applications. Materials 2010; 3:999-1014). These methods typically employ a two-step process whereby the crosslinked structure of keratins is broken down by either oxidation or reduction. In these reactions, the disulfide bonds in cystine amino acid residues are cleaved, rendering the keratins soluble. In some embodiments, reactions cleave the disulfide bonds without appreciable disruption of amide bonds.

If one employs an oxidative treatment, the resulting keratins are referred to as “keratoses.” If a reductive treatment is used, the resulting keratins are referred to as “kerateines” (See Scheme 1).

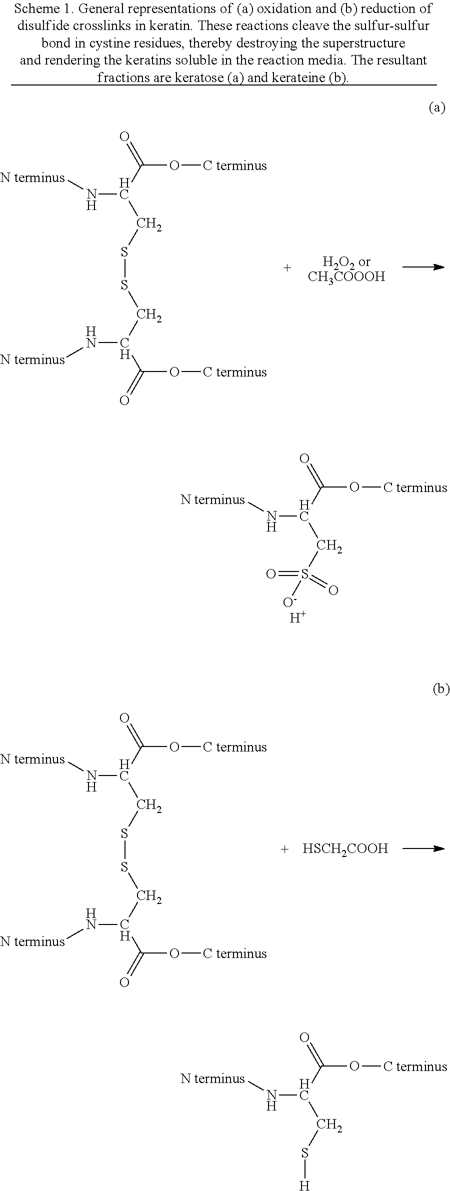

High molecular weight keratins, or “alpha keratins,” (alpha helical), are thought to originate from the microfibrillar regions of the hair follicle, and typically range in molecular weight from about 40-85 kiloDaltons. Low molecular weight keratins, or “gamma keratins,” or keratin-associated proteins (globular), are thought to originate from the matrix regions of the hair follicle, and typically range in molecular weight from about 3-30 kiloDaltons for KAP and 10-15 kiloDaltons for gamma keratins. (See Rouse J G, Van Dyke M E. A review of keratin-based biomaterials for biomedical applications. Materials 2010; 3:999-1014.)

Extracted keratin solutions are known to spontaneously self-assemble at the micron scale (see, e.g., Thomas et al., Int J Biol Macromol 1986; 8:258-64; van de Löcht, Melliand Textilberichte 1987; 10:780-6). Self-assembly results in a highly regular structure with reproducible architectures, dimensionality, and porosity. When the keratin is processed correctly, this ability to self-assemble can be preserved and used to create regular architectures on a size scale conducive to cellular infiltration and/or attachment.

When keratins are hydrolyzed (e.g., with acids or bases), their molecular weight is reduced, and they lose the ability to self-assemble. Therefore, in some embodiments, processing conditions that minimize hydrolysis are preferred.

Many of the keratins can remain trapped within the cuticle's protective structure, so a second-step using a denaturing solution is typically employed to effect efficient extraction of the cortical proteins (alternatively, in the case of reduction reactions, these steps can be combined). This step may use solutions such as urea, transition metal hydroxides, surfactant solutions, and combinations thereof. Preferred methods are aqueous solutions of tris(hydroxymethyl)-aminomethane in concentrations between 0.1 and 1.0M, and urea solutions between 0.1 and 10M,

Crude (unfractionated) extracts of keratins, regardless of redox state, can be further refined into matrix (KAP and gamma), alpha, and/or charged (acidic or basic) fractions by a variety of methods such as isoelectric precipitation, dialysis, or high performance liquid chromatography (HPLC), as desired. In a crude extract, the alpha fraction begins to precipitate below pH 6 and is essentially completely precipitated by pH 4.2.

In some embodiments, KAP co-precipitate with the alpha fraction, thereby producing an alpha/KAP mixture. (see Rogers et al., “Human Hair Keratin-Associated Proteins (KAPs),” Int'l ref. cytol. 251:209-263 (2006).

The gamma fraction remains in solution, but can be precipitated by addition of a non-solvent. Non-solvents are water miscible but do not dissolve keratins. A preferred non-solvent is an alcohol such as ethanol. Precipitation of the gamma fraction can be aided by cooling the ethanol and adding the keratin solution dropwise, rather than adding the ethanol to the keratin.

Keratose Production. A preferred method for the production of keratoses is by oxidation with hydrogen peroxide, peracetic acid, or performic acid. A most preferred oxidant is peracetic acid. Preferred concentrations range from 1 to 10 weight/volume percent, the most preferred being approximately 2 w/v %. Those skilled in the art will recognize that slight modifications to the concentration can be made to affect varying degrees of oxidation, with concomitant alterations in reaction time, temperature, and liquid to solid ratio. It has also been discussed by Crewther et al. that performic acid offers the advantage of minimal peptide bond cleavage compared to peracetic acid. However, peracetic acid offers the advantages of cost and availability. A preferred oxidation temperature is between 0 and 100 degrees Celsius. A most preferred oxidation temperature is 37° C. A preferred oxidation time is between 0.5 and 24 hours. A most preferred oxidation time is 10 hours. A preferred liquid to solid ratio is from 5 to 100:1. A most preferred ratio is 20:1. After oxidation, the hair can be rinsed free of residual oxidant using a copious amounts of purified water.

The keratoses may be extracted from the oxidized hair using an aqueous solution of a denaturing agent. Protein denaturants are well known in the art, but preferred solutions include urea, transition metal hydroxides (e.g. sodium and potassium hydroxide), ammonium hydroxide, and tris(hydroxymethyl)aminomethane (Trizma® base). A preferred solution is Trizma base in the concentration range from 0.01 to 1M. A most preferred concentration is 0.1M. Those skilled in the art will recognize that slight modifications to the concentration can be made to effect varying degrees of extraction, with concomitant alterations in reaction time, temperature, and liquid to solid ratio. A preferred extraction temperature is between 0 and 100 degrees Celsius. A most preferred extraction temperature is 37° C. A preferred extraction time is between 0.5 and 24 hours. A most preferred extraction time is 2 hours. A preferred liquid to solid ratio is from 5 to 100:1. A most preferred ratio is 40:1. Additional yield can be achieved with subsequent extractions with dilute solutions of Trizma base or purified water. After extraction, the residual solids can be removed from solution by centrifugation and/or filtration.

Residual denaturing agent may be removed by dialysis against purified water or buffer solution. Concentration of the dialysis retentate may be followed by lyophilization or spray drying, resulting in a dry powder mixture of gamma and alpha keratoses as well as KAP. Alternately, an alpha/KAP mixture may be isolated from the crude extract solution by dropwise addition of acid until the pH of the solution reaches approximately 4.2. Preferred acids include sulfuric, hydrochloric, and acetic. A most preferred acid is concentrated hydrochloric acid. Precipitation of the alpha/KAP fraction begins at around pH 6.0 and continues until approximately 4.2. Fractional precipitation can be utilized to isolate different ranges of protein with different isoelectric properties. Precipitated alpha/KAP can be recovered by centrifugation, filtration, or the like. The alpha/KAP mixture is further purified by re-dissolving the solids in a denaturing solution. The same denaturing solutions as those utilized for extraction can be used. However, a preferred denaturing solution is Trizma base. Ethylene diamine tetra acetic acid (EDTA) can be added to complex and remove trace metals found in hair. A preferred denaturing solution is 100 mM tris base with 20 mM EDTA or DI water with 20 mM EDTA, if desired. If the presence of trace metals is not detrimental to the intended application, the EDTA step may be omitted. The alpha/KAP mixture can be re-precipitated from this solution by dropwise addition of hydrochloric acid to a final pH of 4.2. Isolation of the solid may be done by centrifugation, filtration or the like. This process can be repeated several times to further purify the alpha/KAP mixture, if desired, although significant destruction of amide bonds should be avoided according to some embodiments. In another preferred embodiment, the alpha/KAP fraction can be isolated from gamma-keratose by dialysis. Providing a high nominal low molecular weight cutoff membrane such that the gamma passes through the membrane and the alpha/KAP is retained can effect such separation. Preferred membranes are those having nominal low molecular weight cutoffs of 15,000 to 100,000 Da. Most preferred membranes are those having nominal low molecular weight cutoffs of 30,000 and 100,000 Da.

The gamma keratose fraction can be isolated by addition to a water-miscible non-solvent. Suitable non-solvents include ethanol, methanol, acetone, and the like. A most preferred non-solvent is ethanol. To effect precipitation, the gamma keratose solution can be concentrated by removal of excess water. This can be done using vacuum distillation, falling film evaporation, microfiltration, etc. After concentration, the gamma keratose solution is added dropwise to an excess of cold non-solvent. A most preferred method is to concentrate the gamma keratose solution to approximately 10 weight/volume (w/v) % protein and add it dropwise to an 8-fold excess of cold ethanol. The precipitated gamma keratose can be isolated by centrifugation or filtration and dried. Suitable methods for drying include freeze drying (lyophilization), air drying, vacuum drying, or spray drying. A most preferred method is freeze drying. Alternately, the gamma keratose can be isolated by dialysis against purified water or buffer solution. Preferred membranes for dialysis are those having nominal low molecular weight cutoffs between 1,000 and 5,000 Da. Most preferred membranes for dialysis are those having nominal low molecular weight cutoffs of 3,000 and 5,000 Da. This solution can be concentrated by additional dialysis and reduced to a dry powder by lyophilization or spray drying.

Several different approaches to further purification can be employed to keratose solutions (e.g., crude, alpha or gamma keratose). Care must be taken, however, to choose techniques that lend themselves to keratin's unique solubility characteristics. One of the most simple separation technologies is isoelectric precipitation. Another general method for separating keratins is by chromatography. Several types of chromatography can be employed to fractionate keratin solutions including size exclusion or gel filtration chromatography, affinity chromatography, isoelectric focusing, gel electrophoresis, ion exchange chromatography, and immunoaffinity chromatography. These techniques are well known in the art and are capable of separating compounds, including proteins, by the characteristics of molecular weight, chemical functionality, isoelectric point, charge, or interactions with specific antibodies, and can be used alone or in any combination to affect high degrees of separation and resulting purity.

A preferred purification method is ion exchange (IEx) chromatography. IEx chromatography is particularly suited to protein separation owning to the amphiphilic nature of proteins in general and keratins in particular. Depending on the starting pH of the solution, and the desired fraction slated for retention, either cationic or anionic IEx (CIEx or AIEx, respectively) techniques can be used. For example, at a pH of 7 and above, both gamma and alpha/KAP keratose fractions are soluble and above their isoelectric points. As such, they are anionic and can be bound to an anionic exchange resin. However, if the pH is below approximately 6, the alpha in the alpha/KAP fraction will not bind to the resin and instead passes through a column packed with such resin. A preferred solution for AIEx chromatography is alpha/KAP solution, isolated as described previously, in weak buffer solution at a concentration between 0 and 5 weight/volume %. A preferred concentration is approximately 2 w/v %. It is preferred to keep the ionic strength of the solution initially quite low to facilitate binding to the AIEx column. This is achieved by using a minimal amount of acid to titrate a purified water solution of the keratin to between pH 5.3 and 6. A most preferred pH is 5.3. This solution can be loaded onto an AIEx column such as DEAE-Sepharose or Q-Sepharose, or processed in bulk without the use of a column. The solution that passes through the column can be collected and further processed as described previously to isolate a fraction of alpha powder.

The basic fraction (including KAP) binds readily due to its lower isoelectric point, and can be washed off the column using salting techniques known in the art. A preferred elution medium is sodium chloride solution. A preferred concentration of sodium chloride is between 0.1 and 2M. A most preferred concentration is 2M. The pH of the solution is preferred to be between 6 and 12. A most preferred pH is 11. In order to maintain stable pH during the elution process, a buffer salt can be added. A preferred buffer salt is Trizma base. A preferred concentration of Trizma base is 100 mM. Those skilled in the art will recognize that slight modifications to the salt concentration and pH can be made to affect the elution of keratin fractions with differing properties. It is also possible to use different salt concentrations and pH's in sequence, or employ the use of salt and/or pH gradients to produce different fractions. Regardless of the approach taken, however, the column eluent can be collected and further processed as described previously to isolate purified fractions of alpha-keratose powders.

A complimentary procedure is also feasible using CIEx techniques. Namely, the alpha/KAP solution can be added to a cation exchange resin such as SP Sepharose (strongly cationic) or CM Sepharose (weakly cationic), and the basic (KAP) fraction collected with the pass through. The retained alpha fraction can be isolated by salting as previously described.

Kerateine Production. Similar to the methods described above for extraction and purification of keratoses, kerateines can be produced by reduction of hair fibers with thioglycolic acid or beta-mercaptoethanol. A most preferred reductant is thioglycolic acid (TGA). Preferred concentrations range from 0.1 to 10M, the most preferred being approximately 1.0M or 0.5M. Those skilled in the art will recognize that slight modifications to the concentration can be made to effect varying degrees of reduction, with concomitant alterations in pH, reaction time, temperature, and liquid to solid ratio. A preferred pH is between 9 and 11. A most preferred pH is 10.2. The pH of the reduction solution is altered by addition of base. Preferred bases include transition metal hydroxides and ammonium hydroxide. A most preferred base is sodium hydroxide. The pH adjustment is affected by dropwise addition of a saturated solution of sodium hydroxide in water to the reductant solution. A preferred reduction temperature is between 0 and 100 degrees Celsius. A most preferred reduction temperature is 37° C. A preferred reduction time is between 0.5 and 24 hours. A most preferred reduction time is 12 hours. A preferred liquid to solid ratio is from 5 to 100:1. A most preferred ratio is 20:1. Unlike the previously described oxidation reaction, reduction is carried out at basic pH. That being the case, keratins are highly soluble in the reduction media and are expected to be extracted. The reduction solution may therefore be combined with the subsequent extraction solutions and processed accordingly.

Reduced keratins are not as hydrophilic as their oxidized counterparts. As such, reduced hair fibers will not swell and split open as will oxidized hair, resulting in relatively lower yields. Another factor affecting the kinetics of the reduction/extraction process is the relative solubility of kerateines. The relative solubility rankings in water, from most to least soluble, is gamma-keratose>alpha-keratose>gamma-kerateine>alpha-kerateine. Consequently, extraction yields from reduced hair fibers are not as high. This being the case, subsequent extractions are conducted with additional reductant plus denaturing agent solutions. Typical solutions for subsequent extractions include TGA plus urea, TGA plus Trizma base, or TGA plus sodium hydroxide. After extraction, crude fractions of alpha/KAP and gamma kerateine can be isolated using the procedures described for keratoses. However, precipitates of gamma and alpha/KAP kerateine re-form their cystine crosslinks upon exposure to oxygen. Precipitates should, therefore, preferably be re-dissolved quickly so as to avoid insolubility during the purification stages, or precipitated in the absence of oxygen.

Purification of kerateine solutions can be conducted similar to those described for keratoses. Those skilled in the art will recognize that the chemical nature of kerateines varies from that of keratoses, primarily in the fate of pendant sulfur groups that will alter chemical properties such as isoelectric points. As such, modifications in the conditions for separation techniques such as ion exchange chromatography are needed for optimization.

All forms of keratin (crude mixtures as well as fractionated materials) have demonstrated an interesting and unexpected ability to change the expression of certain genes in differentiating MSC. Using these different keratins, MSC from a variety of tissues can be exposed to the biomaterial in different forms in both culture systems and in vivo. Different stem and progenitor cells can be used depending on the desired application.

Keratin can be added to the media and the cells seeded onto coated or uncoated cultureware. Alternatively, keratins can be coated onto cell cultureware such as polystyrene dishes or flasks using techniques known in the art. For example, incubation of the cultureware with a dilute solution of keratin (e.g., 100-200 micrograms per mL) can be used. If better adhesion of the coating is desired, a silane coupling agent can be used. Alternatively, thin gel coatings of keratin can be used to provide a three-dimensional matrix to the cells. MSC can then be seeded onto the keratin substrate and subjected to the differentiation protocol.

Differentiation protocols are known in the art. See, e.g., Jaiswal N, Haynesworth S E, Caplan A I, Bruder S P J. Osteogenic differentiation of purified, culture-expanded human mesenchymal stem cells in vitro. Cell Biochem 1997;64(2):295-312 for osteogenesis; Mackay A M, Beck S C, Murphy J M, Barry F P, Chichester C O, Pittenger M F. Chondrogenic differentiation of cultured human mesenchymal stem cells from marrow. Tissue Eng 1998; 4(4):415-28 for chondrogenesis.

In some embodiments, the keratin derivative comprises, consists or consists essentially of a particular fraction or subfraction of keratin. The derivative in some embodiments may comprise, consist or consist essentially of at least 80, 90, 95 or 99 percent by weight of the fraction or subfraction (or more).

In some embodiments, the keratin derivative comprises, consists of, or consists essentially of acidic and/or basic, alpha and/or gamma keratose, where the keratose comprises, consists of or consists essentially of at least 80, 90, 95 or 99 percent by weight of acidic and/or basic, alpha and/or gamma keratose (or more). In some embodiments, the keratin derivative comprises, consists of, or consists essentially of alpha/KAP keratose, where the keratose comprises, consist of, or consists essentially of at least 80, 90, 95 or 99 percent by weight of alpha/KAP keratose (or more).

In some embodiments, the keratin derivative comprises, consists of, or consists essentially of acidic and/or basic, alpha and/or gamma kerateine, where the kerateine comprises, consists of or consists essentially of at least 80, 90, 95 or 99 percent by weight of acidic and/or basic, alpha and/or gamma kerateine (or more). In some embodiments, the keratin derivative comprises, consists of or consists essentially of alpha/KAP kerateine, where the kerateine comprises, consist of, or consists essentially of at least 80, 90, 95 or 99 percent by weight of alpha/KAP keratose (or more).

The basic alpha keratose is preferably produced by separating basic alpha keratose from a mixture comprising acidic and basic alpha keratose, e.g., by ion exchange chromatography, and optionally the basic alpha keratose has an average molecular weight of from 10 to 100, 120, 200 or 250 kiloDaltons. More preferably, the average molecular weight is from 30 or 40 to 90, 100, 120, 150, 200, 250 or 500 kiloDaltons. Optionally, but in some embodiments preferably, the process further comprises the steps of re-dissolving the basic alpha-keratose in a denaturing and/or buffering solution, optionally in the presence of a chelating agent to complex trace metals, and then re-precipitating the basic alpha keratose from the denaturing solution. It will be appreciated that the composition preferably contains not more than 5, 2, 1, or 0.1 percent by weight of acidic alpha keratose, or less.

The acidic alpha keratose may be produced by a reciprocal of the foregoing technique: that is, by separating and retaining acidic alpha keratose from a mixture of acidic and basic alpha keratose, e.g., by ion exchange chromatography, and optionally the acidic alpha keratose has an average molecular weight of from 10 to 100, 120, 200, 250 or 500 kiloDaltons. More preferably, the average molecular weight is from 30 or 40 to 90, 100, 120, 150, 200, 250 or 500 kiloDaltons. Optionally, but in some embodiments preferably, the process further comprises the steps of re-dissolving the acidic alpha-keratose in a denaturing solution and/or buffering solution, optionally in the presence of a chelating agent to complex trace metals, and then re-precipitating the basic alpha keratose from the denaturing solution. It will be appreciated that the composition preferably contains not more than 5, 2, 1, or 0.1 percent by weight of basic alpha keratose, or less.

Basic and acidic fractions of other keratoses (e.g., KAP and gamma keratose) can be prepared in like manner as described above for basic and acidic alpha keratose.

Basic alpha kerateine is preferably produced by separating basic alpha kerateine from a mixture of acidic and basic alpha kerateine, e.g., by ion exchange chromatography, and optionally the basic alpha kerateine has an average molecular weight of from 10 to 100, 120, 200, 250 or 500 kiloDaltons. More preferably, the average molecular weight is from 30 or 40 to 90, 100, 120, 150, 200, 250 or 500 kiloDaltons. Optionally, but preferably, the process further includes the steps of re-dissolving the basic alpha-kerateine in a denaturing and/or buffering solution, optionally in the presence of a chelating agent to complex trace metals, and then re-precipitating the basic alpha kerateine from the denaturing solution. It will be appreciated by those of skill in the art that the composition preferably contains not more than 5, 2, 1, or 0.1 percent by weight of acidic alpha kerateine, or less.

The acidic alpha kerateine may be produced by a reciprocal of the foregoing technique; that is, by separating and retaining acidic alpha kerateine from a mixture of acidic and basic alpha kerateine, e.g., by ion exchange chromatography, and optionally the acidic alpha kerateine has an average molecular weight of from 5 or 10 to 100, 120, 200, 250 or 500 kiloDaltons. More preferably, the average molecular weight is from 30 or 40 to 90, 100, 120, 150, 200, 250 or 500 kiloDaltons. Optionally, but preferably, the process further comprises the steps of re-dissolving the acidic alpha-kerateine in a denaturing and/or buffering solution), optionally in the presence of a chelating agent to complex trace metals, and then re-precipitating the basic alpha kerateine from the denaturing solution. It will be appreciated that the composition preferably contains not more than 5, 2, 1, or 0.1 percent by weight of basic alpha kerateine, or less.

Basic and acidic fractions of other kerateins (e.g., KAP and gamma kerateine) can be prepared in like manner as described above for basic and acidic alpha kerateine. Gamma keratins are typically precipitated in a non-solvent such as ethanol.

As used herein, “acidic” keratins are those keratins that are protonated at a predetermined pH such that they carry a net positive charge; “basic” keratins are those keratins that are de-protonated at a predetermined pH such that they carry a net negative charge. The Keratin Associated Proteins (KAP) as used herein carry a negative charge at the predetermined pH and bind to an anionic exchange resin, and thus in some embodiments is included in the basic keratin fractions taught herein. In some embodiments, the predetermined pH is between 5 and 7. In some embodiments, the pH is 6. For example, in some embodiments, keratose or kerateine is separated into acidic and basic fractions (e.g., by ion exchange chromatography) performed at a solution pH of 6, with the resulting acidic fraction including those keratins having a net positive charge at pH 6, and the basic fraction including those keratins having a net negative charge at pH 6. Likewise, for separation at a predetermined pH of 5.3, the acidic fraction will include those keratins having a net positive charge at pH 5.3 and the basic fraction will include those keratins having a net negative charge at pH 5.3.

Those skilled in the art will recognize that the predetermined pH is selected to effect the best separation between acidic and basic proteins based upon their isoelectric points (see, e.g.,

Table 1), though solubility at that pH should also be considered. When the pH of the solution is between the isoelectric point of these acidic and basic keratin fractions, basic keratin proteins will be de-protonated to have a net negative charge and bind to an anionic media (e.g., DEAE-Sepharose or Q-Sepharose (anion exchange)), while the acidic proteins will be protonated to have a net positive charge and pass through the column, thereby effecting separation.

Further discussion of keratin preparations are found in U.S. Patent Application Publication 2009/0004242 (Van Dyke), which is incorporated by reference herein in its entirety.

In some embodiments, mixtures of a keratose and a kerateine are provided. Because kerateine is absorbed more slowly than keratose in the body, providing a mixture of the two may be useful in controlling the absorption rate in vivo. Preferred ratios according to some embodiments range from 1:10 to 10:1 keratose:kerateine, with most preferred ranging from 1:10 or 1:5 to 1:1 keratose:kerateine.

Formulations. Dry powders may be formed of keratin preparations as described above in accordance with known techniques such as freeze drying (lyophilization). In some embodiments, compositions of the invention may be produced by mixing such a dry powder composition form with an aqueous solution to produce a composition having water or an electrolyte solution with a keratin solubilized therein. The mixing step can be carried out at any suitable temperature, typically room temperature, and can be carried out by any suitable technique such as stirring, shaking, agitation, etc. The salts and other constituent ingredients of the electrolyte solution (e.g., all ingredients except the keratin derivative and the water) may be contained entirely in the dry powder, entirely within the aqueous composition, or may be distributed between the dry powder and the aqueous composition. For example, in some embodiments, at least a portion of the constituents of the electrolyte solution is contained in the dry powder.

The formation of a composition including keratin materials such as described above can be carried out in accordance with techniques long established in the field or variations thereof that will be apparent to those skilled in the art. In some embodiments, the keratin preparation is dried and rehydrated prior to use. See, e.g., U.S. Pat. No. 2,413,983 to Lustig et al., U.S. Pat. No. 2,236,921 to Schollkipf et al., and U.S. Pat. No. 3,464,825 to Anker. In some embodiments, lyophilized material is rehydrated with a suitable solvent, such as water or phosphate buffered saline (PBS). The material can be sterilized, e.g., by ‘y-irradiation (800 had) using a ⁶⁰Co source. Other suitable methods of forming keratin matrices include, but are not limited to, those found in U.S. Pat. No. 6,270,793 (Van Dyke et al.), U.S. Pat. No. 6,274,155 (Van Dyke et al.), U.S. Pat. No. 6,316,598 (Van Dyke et al.), U.S. Pat. No. 6,461,628 (Blanchard et al.), U.S. Pat. No. 6,544,548 (Siller-Jackson et al.), and U.S. Pat. No. 7,01,987 (Van Dyke).

In some embodiments, keratin-containing compositions are sterile filtered and processed aseptically, or terminally sterilized using ethylene oxide, e-beam, gamma, or other low temperature method (i.e. <50° C.).

The composition may be aseptically packaged in a suitable container, such as a flexible polymeric bag or bottle, or a foil container, or may be provided as a kit of sterile dry powder in one container and sterile aqueous solution in a separate container for mixing just prior to use.

When provided packaged in a sterile container, in some embodiments the composition preferably has a shelf life of at least 4 or 6 months (up to 2 or 3 years or more) at room temperature, prior to substantial loss of viscosity (e.g., more than 10 or 20 percent) and/or structural integrity of the keratin composition.

The composition may be provided in a precursor solution. For example, keratin-containing precursor solution can be provided in a glass ampule ready to use directly or after dilution by the user. In the case of kerateine compositions, which can re-crosslink in the presence of oxygen in air, a sterile precursor solution in a sealed ampule under an inert atmosphere (e.g., nitrogen) can be provided.

Formulations of the invention include those for injection or implantation. In one embodiment, administration is carried out either by simple injection into the tissue, area surrounding the tissue of interest (e.g., synovial space), or into a tissue defect needing repair. In another embodiment, administration is carried out as a graft to an organ or tissue to be augmented. In some embodiments, cells are administered by injection of the cells (e.g., in a suitable carrier) directly into the tissue of a subject (e.g., bone or cartilage tissue).

In some embodiments, gels will form in the range of 3, 4 or 5, to 8, 10, 12 or 15 weight percent. The concentration can be altered and/or the precursor solution diluted during use depending on the stiffness of the gel desired, with lower concentration of solids forming a less stiff gel and more easily injected, and high solids more stiff and more easily handled and implanted manually. In some embodiments, 3, 4 or 5 to 6, 7 or 8 weight percent solids produce softer gels and less cell growth. In some embodiments, higher solids such as 8, 9 or 10, to 11, 12, 13, 14 or 15% are stiffer and provide better cell attachment and growth. The balance of these solutions according to some embodiments may be water, saline, or other biocompatible solution (and may depend on the need for osmotically balancing the resulting gel).

In some embodiments, cells are administered in a therapeutically effective amount. The therapeutically effective dosage of cells will vary somewhat from subject to subject, and will depend upon factors such as the age, weight, and condition of the subject and the route of delivery. Such dosages can be determined in accordance with procedures known to those skilled in the art. In general, in some embodiments, a dosage of 1×10⁵, 1×10⁶ or 5×10⁶ up to 1×10⁷, 1×10⁸ or 1×10⁹ cells or more per subject may be given, administered together at a single time or given as several subdivided administrations. In other embodiments a dosage of between 1-100×10⁸ cells per kilogram subject body weight can be given, administered together at a single time or given as several subdivided administrations. Of course, follow-up administrations may be given, if needed or desired. In some embodiments, the cells may be pre-differentiated into osteogenic or chondrogenic cells prior to administration. In other embodiments, undifferentiated cells may be contained in the keratin composition, with or without other differentiating factors. In yet another embodiment, the keratin, itself, may be administered and cause endogenous cells to differentiate.

In further embodiments, if desired or necessary, the subject may be administered an agent for inhibiting transplant rejection of the administered cells, such as rapamycin, azathioprine, corticosteroids, cyclosporin and/or FK506, in accordance with known techniques. See, e.g., R. Caine, U.S. Pat. Nos. 5,461,058, 5,403,833 and 5,100,899; see also U.S. Pat. Nos. 6,455,518, 6,346,243 and 5,321,043.

As used herein, the administration of two or more compositions (inclusive of keratin compositions and cells) “in combination” means that the two agents are administered closely enough in time that the presence of one alters the biological effects of the other. The two may be applied simultaneously (concurrently or contemporaneous) or sequentially. Administrations according to some embodiments may be within a period of time that ranges from minutes (e.g., 1, 5, 10, 30, 60, or 90 minutes or more) to days (e.g., 1, 2, 5, 8 or 10 or more days), as appropriate for efficacious treatment.

Simultaneous, concurrent or contemporaneous administration may be carried out by mixing the compositions prior to administration, or by administering the compositions at the same point in time but at different sites of the body or using different types of administration, or applied at times sufficiently close that the results observed are indistinguishable from those achieved when the compositions are administered at the same point in time.

Sequential administration may be carried out by administering one composition at some point in time prior to administering another composition, such that the prior administration enhances the effects of the subsequent administration (e.g., percentage or yield of desired tissues formed). In some embodiments, a keratin composition is administered at some point in time prior to and/or after the initial administration of cells. Optionally, a keratin composition may be administered again at some point in time after the administration of cells.

The present invention is explained in greater detail in the following non-limiting Examples.

### EXAMPLES

### Example 1

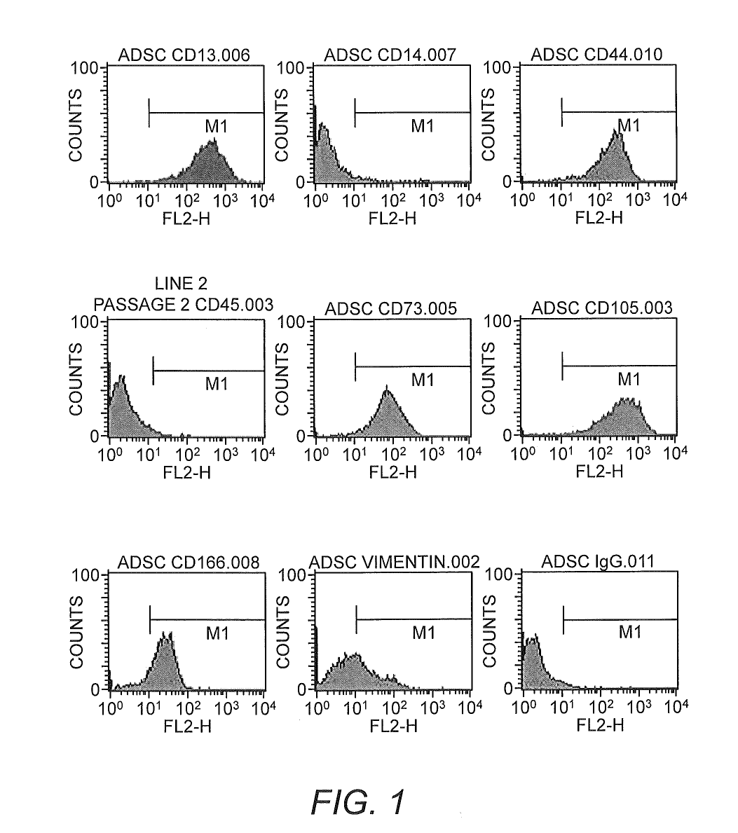

Human adipose-derived stem cells (ADSC) were isolated successfully from adipose tissue by collagenase digestion. More than 98% of the expressed CD13, CD44, CD73 and CD105. 88% of them expressed CD106 and 43.76% of them expressed vimentin. Most cells were negative for the hematopoietic markers CD14 and CD45 (FIG. 1). The cells grown in control media showed characteristic fibroblast-like morphology.

After surface marker characterization, human ADSCs were differentiated towards an osteogenic lineage by culturing them at a density of 3,000 cells/cm² in basic osteogenic differentiation media containing DMEM low glucose culture, 10% FBS, 1% antibiotic/antimycotic, 10⁻⁷M dexamethasone, 50 mM ascorbic acid and 10 mM β-glycerophosphate disodium salt pentahydrate for 21 days. This basic osteogenic media (represented as 10⁻⁷ Dex) was supplemented with the addition of keratose fractions 0.01 mg/ml alpha (A-KOS), 0.03 mg/ml gamma (G-KOS) or 0.01 mg/ml crude (C-KOS). Total RNA was harvested on days 0, 8, 15 and 21. Quantitative real-time PCR assays were performed using osteogenic-specific primers (osteocalcin, Alk.phos, coll typela, Runx2, DLX5) and adipogenic-specific primers (PPARγ) to detect osteogenic or adipogenic marker mRNA expression, respectively.

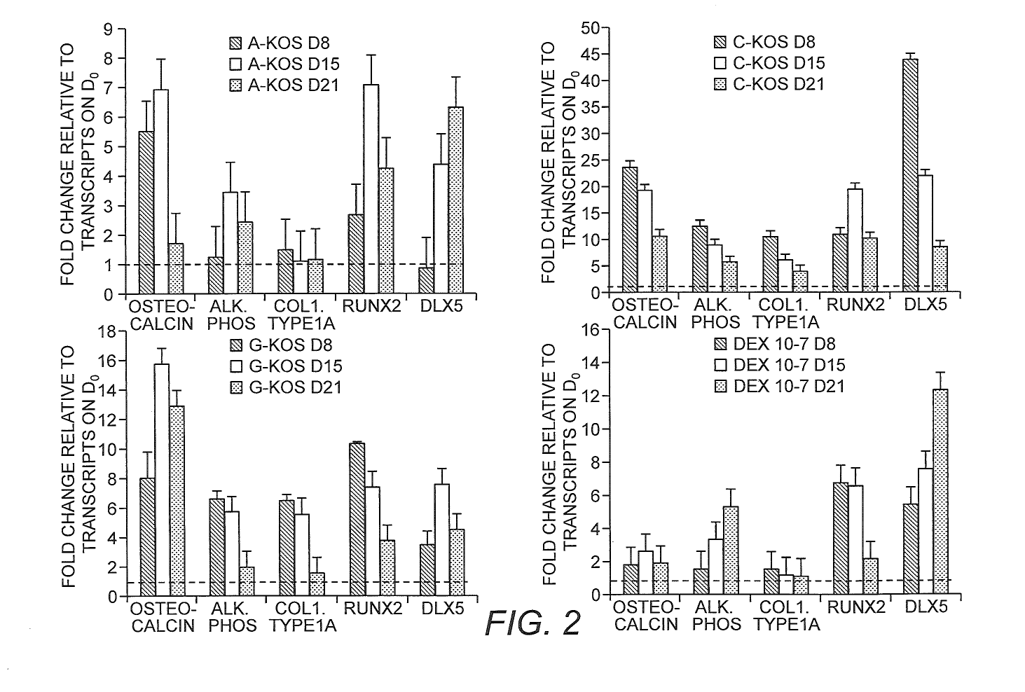

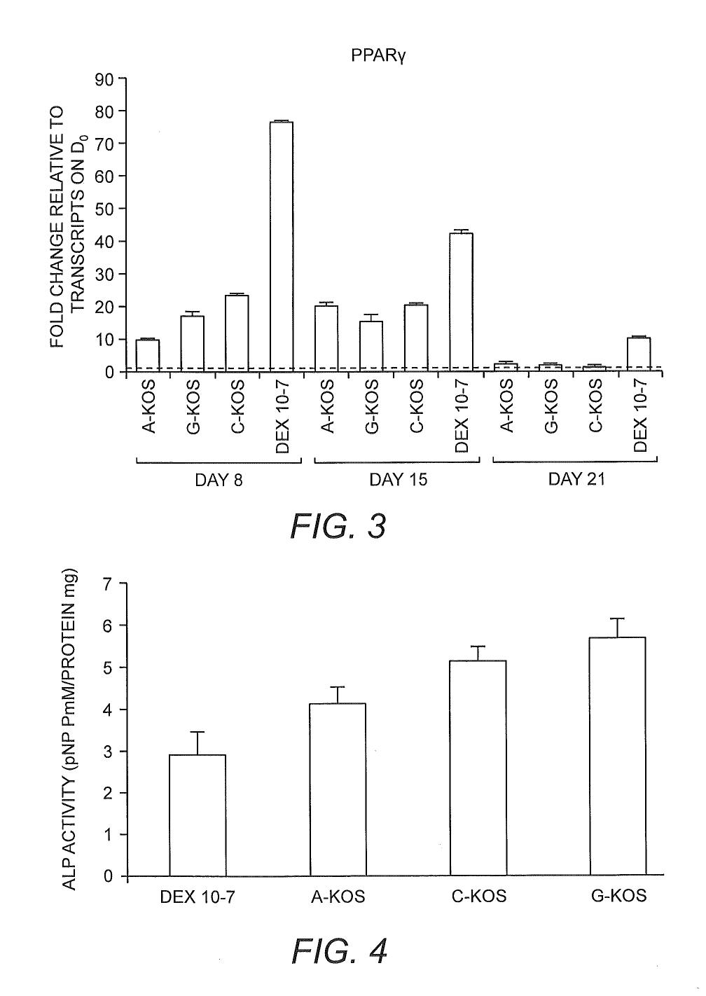

Gene expression profiling data demonstrate the ability of all keratose fractions to upregulate osteoblast markers, especially at earlier time points at days 8 and 15 (FIG. 2). The addition of keratose fractions in the basic osteogenic media also induced a significant (p<0.01) down-regulation of PPARγ expression only for the day 8 and 15 time points (FIG. 3), demonstrating their ability to selectively regulate the fate of ADSCs.

The activity of alkaline phosphatase (AP) was determined by quantifying the conversion of para-nitrophenyl phosphate (p-Npp) to para-nitrophenol (p-Np). Samples (n=6 per group per treatment) were prepared by lysing cells with 0.1% Triton X-100 and incubating them at 37° C. for 30 minutes. The absorbance was read at 405nm with a microplate spectrophotometer. The AP activity was normalized to the protein concentration of each sample, as measured by BCA protein assay (Pierce) and expressed as nmol/min/mg protein.

These data show an increase in alkaline phosphatase activity in keratin treated cultures (FIG. 4), an indication of their osteoblast-like phenotype.

Calcium deposition of differentiated ADSCs was determined by Alizarin Red staining. In brief, the cells were rinsed with calcium and phosphate-free saline solution and fixed with ice-cold 70% ethanol for 10min. After a brief wash with water, the cells were stained for 3 min 0.5% Alizarin Red S solution (pH 4.2) at room temperature. A quantitative destaining procedure followed, where stained cultures were destained using 10% (w/v) cetylpyridinium chloride in 100 mM sodium phosphate (pH 7.0) for 3-4hrs at room temperature and the Alizarin Red S concentrations were determined by absorbance measurement at 540 nm.

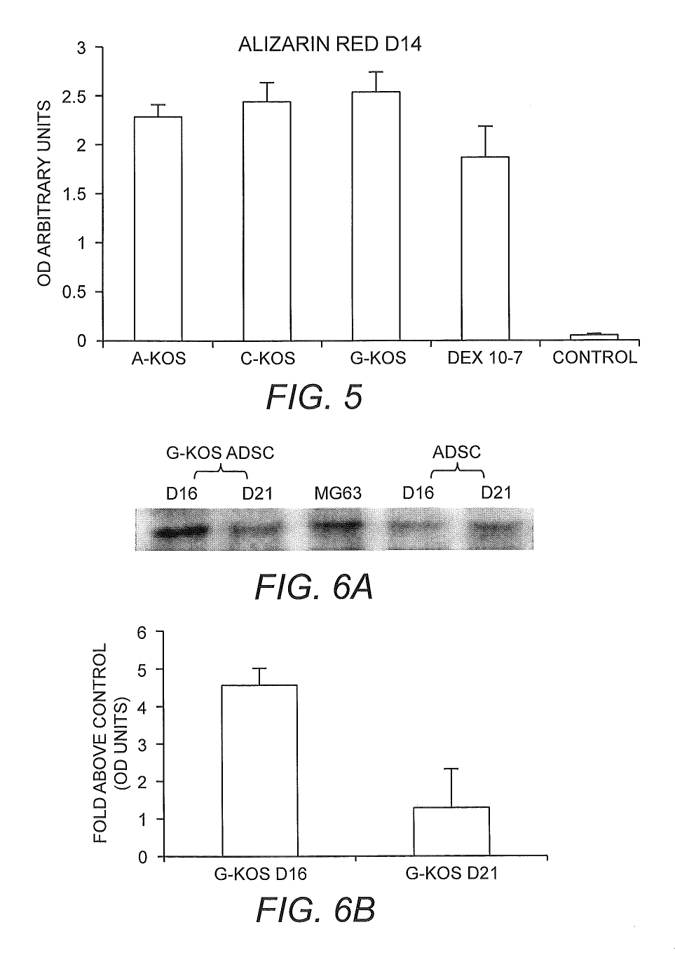

These data show more calcium staining in keratin treated cultures, indicating the deposition of more mineralized matrix by the cells (FIG. 5). The protein expression of osteocalcin in the differentiated ADSCs was investigated by immunoblot analysis of the collected cell culture media. Protein concentrations were determined by Bradford assay. Sixty micrograms of protein from conditioned medium collected from ADSC cultures or MG63 osteosarcoma cell line were loaded onto a 4-12% Criterion XT Bis-Tris Gel. After electrophoresis, the SDS-PAGE separated proteins were transferred to a PVDF membrane (Amersham Pharmacia Biotech). The membrane was blocked with blocking buffer (1× Blocking Buffer, Ambion) for lhr and incubated with rabbit antibody against human osteocalcin (1:500 dilution) in 0.03% nonfat milk in PBS-Tween20 overnight at 4° C. with mild shaking, then the membrane was washed with PBST (6×10 min) and incubated with goat anti-rabbit IgG conjugated with horseradish peroxidase at 1:3000 in PBST for lh. The blots were washed and processed using an ECL Kit (SuperSignal West Femto Maximum Sensitivity Substrate) and exposed to image analysis. Intensities of bands were measured using ImageJ densitometry software. The MG63 cell line is used as a positive control. The cells are grown to confluency and induced to osteocalcin release by the addition of 10⁻⁸ M Vitamin D₃.

A representative experiment is shown in FIG. 6. These data confirm that the expression of the osteocalcin gene, a marker of osteoblast phenotype, results in the secretion of the protein.

In general, all keratin fractions tested have the ability to up-regulate osteogenic genes and down-regulate PPARγ.

### Example 2

Culture of mesenchymal stem cells on kerateine substrates. Keratins are intermediate filament cytoskeletal proteins that form stable network structures, and thus have the potential to be used as substrates for cell attachment in the development of tissue-engineered constructs. Adult mesenchymal stem cells (MSCs) derived from bone marrow (BM-MSCs) and adipose tissue (AD-MSCs) are commonly employed for biomaterial seeding since they have the capability to differentiate into multiple connective tissues including bone and adipose to replace the targeted damaged structure.

The reduced form of keratins called kerateines can assemble through disulfide bonding and such assembled kerateines can support a variety of cells, particularly those with fibroblast morphologies. It is therefore thought that the fibroblastic MSCs can attach and thrive on kerateines.

To test the hypothesis, BM-MSCs and AD-MSCs were first seeded on kerateine coating (K2D) and kerateine thin gel/film (K3D). Coatings were produced by incubating culture plates in a dilute solution of kerateine overnight, aspirating the excess solution, and rinsing the coating with buffer solution prior to cell seeding. Thin films were prepared by pipetting a 4 weight percent solution of kerateine into tissue culture plates and allowing the material to crosslink by overnight exposure to air (with a lid on the plate) at 37 degrees C. The gels were conditioned with cell media prior to seeding. BM-MSCs and AD-MSCs were also seeded on control surfaces including uncoated plasma-treated tissue culture plastic (UNC), gelatin coating (GEL), and Matrigel™ coating (BD Biosciences) (MAT). Cell behavior (attachment, viability, and proliferation) on the five different substrates were quantified. Attachment ratios were obtained by the number of adhered cells after 6 h of incubation divided by the total number of seeded cells. Cell viabilities were measured using Live/Dead® microscopy assay (Invitrogen). Cell proliferation results determined by growth curves and doubling times were obtained by treating the cells with the MTS reagent at multiple time points over a 1-week span. MSCs were also assessed for their surface markers (+CD29, 44, 73, 90, 105; −CD14, 34, 45) at different passages using flow cytometry. MSCs were differentiated for osteogenesis (+dexamethasone, vitamin C, and glycerol-2-phosphate) and adipogenesis (+dexamethasone, IBMX, indomethacin, and insulin) for 3 weeks and subsequently stained using Alizarin Red S and Oil Red O, respectively. The stained images were quantified and normalized against the number of cells (DAPI staining), Finally, using quantitative real-time PCR (qRT-PCR), osteogenic (osteocalcin and RUNX2) and adipogenic (PPARγ and lipoprotein lipase) gene expressions were evaluated. Undifferentiated cells were used as controls for both staining and PCR experiments.

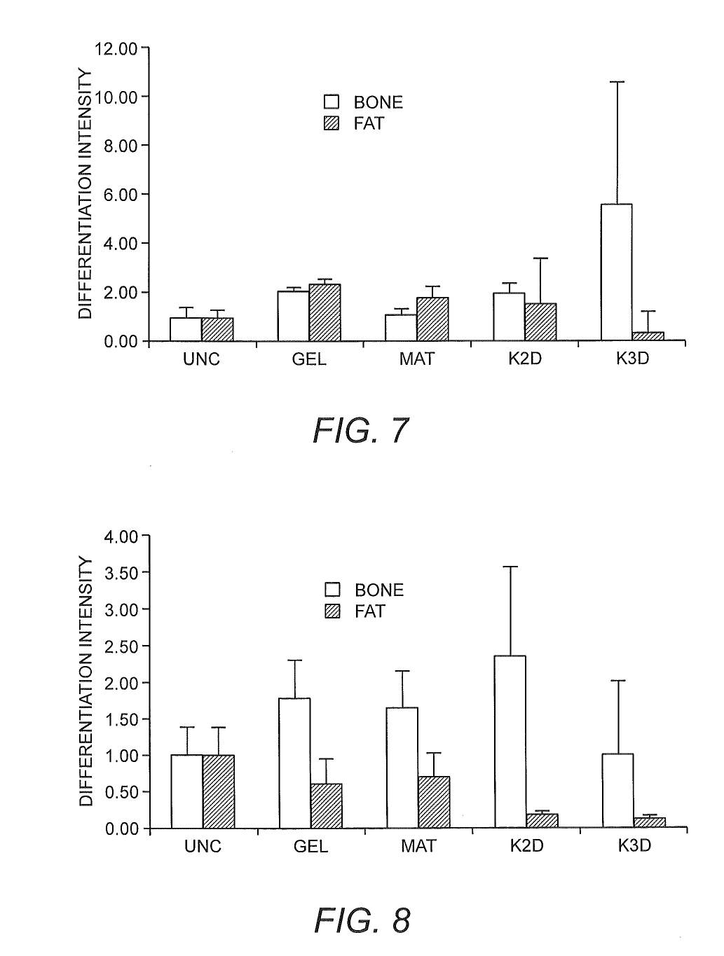

Kerateine substrates (K2D and K3D) (alpha+KAP, dialyzed using a 100 kDa low molecular weight cutoff membrane) as wells as the controls (untreated or “UNC”, gelatin or “GEL”, and Matrigel® “MAT”) were able to support initial adhesion of cells at >94%. After the attachment, MSCs from both adipose tissue and bone marrow remained highly viable over time. They proliferated on all surfaces with doubling time ranging from 2.5-3.5 days. Kerateine substrates generally allowed a slower rate of proliferation compared to the controls, and it was found that growth on K2D>K3D. Kerateine films with softer consistency (at 4 weight percent) also provided less cell growth. Positive and negative stem cell-surface markers were retained on cells on kerateins. Under differentiation conditions (i.e., use of osteogenic and adipogenic induction media), BM-MSCs on kerateine substrates appeared to induce higher osteogenic differentiation but fewer adipocytes when induced with the adipogenic media. The differentiation capacities on UNC, GEL, and MAT all appear similar compared to the UNC control (FIG. 7). AD-MSCs generally differentiate into osteoblasts more on GEL, MAT, K2D, and K3D than UNC. However, for adipogenic differentiation only the kerateine substrates provided a down regulatory response compared to the uncoated control (FIG. 8). qRT-PCR is performed to confirm the staining in which the kerateine substrates provided less capacity to differentiate into adipocytes.

Results from this study support the use of kerateine biomaterial for bone regeneration to drive the repair stem cells towards the osteogenic lineage and avoid the adipogenic route.

The foregoing is illustrative of the present invention, and is not to be construed as limiting thereof. The invention is defined by the following claims, with equivalents of the claims to be included therein.

1. A method of treating a bone injury or disease in a subject in need thereof comprising administering in combination to said subject in a treatment effective amount:
a composition comprising a keratin; and
a composition comprising stem cells or progenitor cells.

2. The method of claim 1, wherein said keratin is selected from the group consisting of: keratose, kerateine, and combinations thereof.

3. The method of claim 1, wherein said subject is a human subject.

4. The method of claim 1, wherein said subject is an adult subject.

5. The method of claim 1, wherein said subject is osteopenic or osteoporotic.

6. The method of claim 1, wherein said cells are mesenchymal stem cells.

7. The method of claim 6, wherein said mesenchymal stem cells are harvested from bone marrow or adipose tissue.

8. The method of claim 6, wherein said cells are adipose derived stem cells.

9. The method of claim 1, wherein said cells are allogeneic or autologous, and said cells are from a human donor who is at least 40 years old at the time of harvest.

10. The method of claim 1, wherein said keratin is selected from the group consisting of: acidic keratose, acidic kerateine, KAP, and combinations thereof.

11. The method of claim 1, wherein said keratin is selected from the group consisting of: alpha/KAP keratose, gamma keratose, alpha/KAP kerateine, gamma kerateine, and combinations thereof.

12. The method of claim 1, wherein said administering is carried out simultaneously.

13. The method of claim 1, wherein said administering is carried out sequentially.

14. A method of differentiating stem cells into osteogenic cells, comprising:
providing stem cells;
providing a keratin in fluid contact with said stem cells; and
providing conditions conducive to differentiation of said stem cells into osteogenic cells, to thereby differentiate said stem cells into osteogenic cells.

15. The method of claim 14, wherein said keratin is selected from the group consisting of: keratose, kerateine, and combinations thereof.

16. The method of claim 14, wherein said stem cells are mesenchymal stem cells.

17. The method of claim 14, wherein said stem cells are from an adult donor.

18. The method of claim 14, wherein said stem cells are from a donor who is at least 40 years old at the time of harvest.

19. The method of claim 14, wherein said keratin is selected from the group consisting of: acidic keratose, acidic kerateine, KAP, and combinations thereof.

20. The method of claim 14, wherein said keratin is selected from the group consisting of: alpha/KAP keratose, gamma keratose, alpha/KAP kerateine, gamma kerateine, and combinations thereof.

21. The method of claim 14, wherein said stem cells are human.

22. The method of claim 14, wherein said keratin is provided in the form of a media additive.

23. A method of differentiating stem cells into chondrogenic cells, comprising:
providing stem cells;
providing a keratin in fluid contact with said stem cells; and
providing conditions conducive to differentiation of said stem cells into chondrogenic cells,
to thereby differentiate said stem cells into chondrogenic cells.

24. The method of claim 23, wherein said keratin is selected from the group consisting of: keratose, kerateine, and combinations thereof.

25. The method of claim 23, wherein said stem cells are mesenchymal stem cells.

26. The method of claim 23, wherein said stem cells are from an adult donor.

27. The method of claim 23, wherein said stem cells are from a donor who is at least 40 years old at the time of harvest.

28. The method of claim 23, wherein said keratin is selected from the group consisting of: acidic keratose, acidic kerateine, KAP, and combinations thereof.

29. The method of claim 23, wherein said keratin is selected from the group consisting of: alpha/KAP keratose, gamma keratose, alpha/KAP kerateine, gamma kerateine, and combinations thereof.

30. The method of claim 23, wherein said cells are human.

31. The method of claim 23, wherein said keratin is provided in the form of a hydrogel or a media additive.
